(19) United States
(12) Patent Application Publication
(10) Pub. No.: US 2023/0398123 A1
(43) Pub. Date: Dec. 14, 2023

(54) METHODS TO TREAT INFLAMMATORY BOWEL DISEASE

(71) Applicant: SHANGHAI PHARMACEUTICALS HOLDING CO., LTD., Shanghai (CN)

(72) Inventors: Xin NAKANISHI, San Diego, CA (US); Guangxin XIA, Shanghai (CN); Wei SU, Shanghai (CN)

(73) Assignee: SHANGHAI PHARMACEUTICALS HOLDING CO., LTD., Shanghai (CN)

(21) Appl. No.: 18/024,290

(22) PCT Filed: Sep. 3, 2021
(86) PCT No.: PCT/CN2021/116348

§ 371 Date: Mar. 2, 2023

(51) Int. Cl. A61K 31/5377; A61K 45/06; A61P 1/00

(52) U.S. Cl. CPC A61K 31/5377; A61P 1/00; A61K 45/06

(57) ABSTRACT

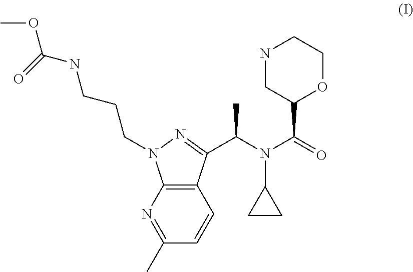

The present invention relates to pharmaceutical methods, compositions, combinations for the treatment and/or prevention of inflammatory bowel diseases (IBD). The invention relates particularly to methods and compositions comprising the compound of Formula (I) or a pharmaceutically acceptable salt thereof, for treating IBD.

### CROSS-REFERENCE TO RELATED APPLICATIONS

This application claims benefit of priority to PCT/CN2020/113466, filed 4 Sep. 2020, the contents of which are incorporated herein for all purposes.

### FIELD OF THE INVENTION

The invention relates to methods for treatment of inflammatory bowel disease (IBD), and compositions and medicaments useful for treating IBD. The methods and compositions are based on data showing that a renin inhibitor of Formula (I) can alleviate symptoms and manifestations of IBD.

### BACKGROUND OF THE INVENTION

Inflammatory bowel disease is often a chronic condition that can dramatically affect quality of life. IBD includes Crohn's disease (CD) and ulcerative colitis (UC). While they are not well understood, it is generally believed they involve excessive or abnormal activation of the mucosal immune system. Current therapies for IBD include anti-inflammatory corticosteroids, aminosalicylates (e.g., mesalamine, balsalazide, olsalazine), immune pathway inhibitors (azathioprine, mercaptopurine, cyclosporine, methotrexate, TNF-alpha inhibitors), and others. However, some patients do not respond to the available therapeutic agents, and some patients respond initially to a known therapeutic regimen, which then loses efficacy. Therefore, there remains a need for new treatment modalities for IBD.

It has been reported recently that activation of the Renin-Angiotensin System (RAS) promotes colitis. Y. Shi, et al., Scientific Reports (Nature) 6, 27552; doi: 10.1038/srep27552 (2016). RenTgMK mice that overexpress active renin from the liver developed more severe colitis than wild-type controls following intrarectal 2,4,6-trinitrobenzene sulfonic acid (TNBS) instillation. More than 50% of the RenTgMK mice died, whereas all the wild-type mice recovered. The RenTgMK mice also exhibited more robust mucosal TH17 and TH1/TH17 responses and more profound colonic epithelial cell apoptosis compared to wild-type controls.

Treatment of these RenTgMK mice with aliskiren, a renin inhibitor administered by intraperitoneal injection, ameliorated this induced colitis in the RenTgMK mice, while treatment with hydralazine, a smooth muscle relaxant that lowers blood pressure similarly to aliskiren, did not affect colitis, demonstrating that colitis relief by the aliskiren treatment is independent of the hypotensive effect that is common to aliskiren and hydralazine.

Aliskiren was the first direct renin inhibitor approved to treat high blood pressure. While it has been used extensively for that purpose, it poses some risk to patients with diabetes and renal impairment due to potential renal toxicity. It also has relatively low bioavailability, only 2.5% (Tekturna® (aliskiren) label), and is complex and expensive to synthesize due to the presence of four chiral centers along an extended linear backbone.

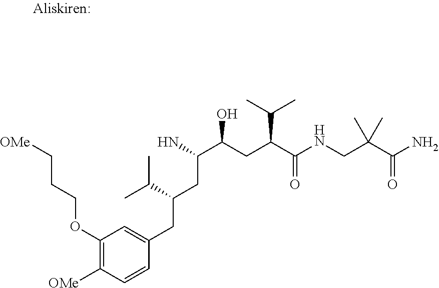 [SMILES: COCCCOc1cc(C[C@@H](C[C@H](N)[C@@H](O)C[C@H](C(=O)NCC(C)(C)C(N)=O)C(C)C)C(C)C)ccc1OC]

The authors of the Shi study acknowledge that their model system is not necessarily applicable to normal metabolic conditions, because the transgenic test animals used are predisposed to amplify the effects of a RAS inhibitor. They note that the findings may not mean that endogenous RAS plays a role in colitis development under ‘normal conditions.’ “The RenTg mouse model is basically an ‘artificial’ system that amplifies the effect of the RAS for investigation. Whether under normal conditions the endogenous RAS plays a role in colitis development needs to be addressed . . . Therefore, it needs to be cautious to generalize our conclusion with regards to the colitogenic effects of the RAS.” Shi at pp. 7-8.

### DISCLOSURE OF THE INVENTION

The present invention provides new IBD treatment methods and compositions using a direct renin inhibitor of Formula (I). This compound has superior bioavailability to aliskiren, and is a more potent as an inhibitor of renin. Data herein demonstrate that the compound of Formula (I) is effective to treat IBD in a model system using both a ‘normal’ (one not genetically predisposed to be especially sensitive to RAS activity) rat and a mouse. In addition, a compound of Formula (I) has been shown to reduce inflammatory cytokine release in colon tissue from human patients with ulcerative colitis. Furthermore, the rat data demonstrate that the compound of Formula (I) is effective to treat IBD when administered orally.

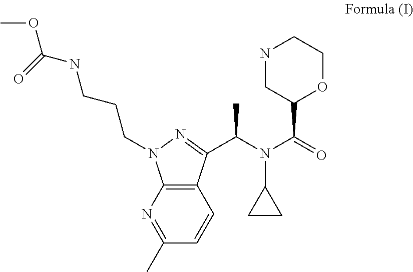

The compound of Formula (I) is a renin inhibitor, but it is not clear whether its effect on IBD is due to inhibition of renin, since data herein show that it inhibits release of some key proinflammatory cytokines, including IL-6; the mechanism of action in IBD has not been explored, and may be multifaceted. However, the methods of the invention are believed to operate by a different mechanism from currently approved IBD therapeutics, thus they can be used where current therapeutics have lost efficacy or they can be combined with current IBD therapeutics to provide new and more effective treatments for patients having IBD.

In one aspect, the present disclosure provides methods to treat inflammatory bowel disease using a compound of Formula (I). Without being bound by theory, the compound has been shown to be a potent direct inhibitor of reninand to reduce the levels of proinflammatory cytokines that may contribute to its effectiveness for treatment of IBD. It has pharmacokinetic properties suitable for therapeutic use via oral administration and it has now shown to be effective for in vivo treatment of inflammatory bowel disease.

Without being bound by theory, it is believed that the compound of Formula (I) treats IBD via a new mechanism of action or combination of mechanisms that can complement current therapies. It can be used along with current IBD therapies, or as an alternative for patients who experience problems with current IBD therapies, or for patients who do not achieve adequate response to current IBD therapies. IBD that can be treated with these methods include Crohn's disease and ulcerative colitis. The methods are useful to treat a subject diagnosed with IBD, e.g., ulcerative colitis or Crohn's disease.

In some embodiments, the compound of Formula (I) is administered orally, typically as a solid dosage form such as a tablet or capsule. Other suitable formulations include a softgel for oral administration, and a suppository for direct introduction into the colon. Administration may be in a single dose or in multiple doses, and a dosage of the compound of Formula (I) may be administered at least once per day, typically in one or two or three tablets or capsules, or it can be administered once every other day, or at least once per week. In some embodiments, a single dosage is administered to a subject in need of treatment for ulcerative colitis or Crohn's disease at least once per day. In other embodiments, a single dosage is administered to the subject twice per day or three times per day. In a preferred embodiment, a dosage is administered twice per day, typically by oral administration.

In another aspect, the invention provides a method as described above, wherein the compound of Formula (I) is administered to a subject who is also being treated with another IBD therapy, which can be selected from, for example, anti-inflammatory corticosteroids, aminosalicylates, and other IBD therapies including, but not limited to:
a) Anti-TNFα agents (e.g., infliximab, adalimumab, certolizumab, golimumab);
b) Sphingosine-1-phosphate (S1P)-receptor modulators (e.g., ozanimod);
c) Anti-adhesion (anti-integrin) agents (e.g., natalizumab, vedolizumab, ertolizumab);
d) IL-12/IL-23 inhibitors (e.g., ustekinumab, risankizumab);
e) Transforming growth-factor beta (TGFβ) inhibitors (e.g., mongersen, pirfenidone);
f) Phosphodiesterase 4 (PDE4) inhibitors (e.g., aprimelast);
g) Janus kinase (JAK)/signal transducers and activators of transcription (STAT) inhibitors (e.g., tofacitinib, filgotinib);
h) Stem-cell transplants (e.g., hematopoietic stem cells, adipose-derived stem cells);
i) Fecal microbiota transplants (FMT);
j) Plasminogen activator inhibitor-1 (PAI-1) inhibitors (e.g., MDI-2268, tiplaxtinin);
k) Aminosalicylates (e.g., mesalamine, balsalazide, olsalazine);
l) Anti-inflammatory corticosteroids; and,
m) Immune pathway inhibitors such as azathioprine, mercaptopurine, cyclosporine, and methotrexate.

In another aspect, the invention provides a solid dosage form comprising a compound of Formula (I), which may be formulated for treating an IBD. The solid dosage form typically contains between 25 mg and 800 mg of the compound of Formula (I) or of a pharmaceutically acceptable salt thereof in a single unit dosage formulated for oral administration. In some such embodiments, the compound of Formula (I) or a pharmaceutically acceptable salt thereof is formulated in a dosage form, such as a tablet, capsule, softgel or suppository, that also comprises at least one additional IBD therapeutic agent selected from anti-inflammatory corticosteroids, aminosalicylates, or other IBD therapeutics such as:
Anti-TNFα agents;
Sphingosine-1-phosphate (S1P)-receptor modulators (e.g., ozanimod);
Anti-adhesion (anti-integrin) agents;
IL-12/IL-23 inhibitors;
Transforming growth-factor beta (TGFβ) inhibitors (e.g., mongersen, pirfenidone);
Phosphodiesterase 4 (PDE4) inhibitors (e.g., aprimelast);
Janus kinase (JAK)/signal transducers and activators of transcription (STAT) inhibitors (e.g., tofacitinib, filgotinib); and,
Plasminogen activator inhibitor-1 (PAI-1) inhibitors (e.g., MDI-2268, tiplaxtinin).

In still another aspect, the present disclosure provides delayed release formulation comprising the compound of Formula (I) or a pharmaceutically acceptable salt thereof for oral administration. Typically, the delayed release formulation is configured or designed to passed through the stomach and into the intestines before it releases most or substantially all of the compound of Formula (I) or a pharmaceutically acceptable salt thereof in the intestine and particularly in the colon of a subject. The invention also provides a method of treating IBD by administering such a delayed release formulation to a subject in need of treatment for an IBD.

In yet another aspect, the present disclosure provides the compound of Formula (I) or a pharmaceutically acceptable salt thereof for the treatment of an inflammatory bowel disease. In some embodiments, the compound of Formula (I) or a pharmaceutically acceptable salt thereof is formulated for oral administration to a subject in need of treatment for an inflammatory bowel disease. In some such embodiments, the compound or its pharmaceutically acceptable salt is formulated as a delayed release formulation designed to pass through the stomach of a recipient before most or substantially all of the compound of Formula (I) or a pharmaceutically acceptable salt thereof is released in the intestinal tract of the recipient. In some such embodiments, the majority of the compound of Formula (I) or a pharmaceutically acceptable salt thereof is released in the colon of the treated subject.

In yet another aspect, the invention provides a method to use the compound of Formula (I) or a pharmaceutically acceptable salt thereof in the manufacture a medicament for use to treat an inflammatory bowel disease. In some such embodiments, the medicament is formulated for oral delivery. In some such embodiments, the medicament is formulated as a delayed release formulation that passes through the stomach of a subject before most or substantially all of the compound of Formula (I) or a pharmaceutically acceptable salt thereof is released in the intestines of the subject.

In yet another aspect, the present disclosure provides for a combination for treating and/or preventing an Inflammatory Bowel Disease, comprising administering the compound of Formula (I) or a pharmaceutically acceptable salt thereof in addition to treating the subject with at least one other IBD therapy, which can be selected from:
a) Anti-TNFα agents (e.g., infliximab, adalimumab, certolizumab, golimumab);
b) Sphingosine-1-phosphate (S1P)-receptor modulators (e.g., ozanimod);
c) Anti-adhesion (anti-integrin) agents (e.g., natalizumab, vedolizumab, ertolizumab);
d) IL-12/IL-23 inhibitors (e.g., ustekinumab, risankizumab);
e) Transforming growth-factor beta (TGFβ) inhibitors (e.g., mongersen, pirfenidone);
f) Phosphodiesterase 4 (PDE4) inhibitors (e.g., aprimelast);
g) Janus kinase (JAK)/signal transducers and activators of transcription (STAT) inhibitors (e.g., tofacitinib, filgotinib);
h) Stem-cell transplants (e.g., hematopoietic stem cells, adipose-derived stem cells);
i) Fecal microbiota transplants (FMT);
j) Plasminogen activator inhibitor-1 (PAI-1) inhibitors (e.g., MDI-2268, tiplaxtinin);
k) Aminosalicylates (e.g., mesalamine, balsalazide, olsalazine);
l) Anti-inflammatory corticosteroids; and,
m) Immune pathway inhibitors such as azathioprine, mercaptopurine, cyclosporine, and methotrexate.

### BRIEF DESCRIPTION OF THE DRAWINGS

FIG. 1 shows body weight of test animals (Wistar rats in a DNBS-induced colitis model) for Example 1.

FIG. 2 shows stool consistency scored over the 7-day test as described, using the area under the curve (AUC) for each group as an index of effect.

FIG. 3 shows macroscopic evaluation of colons in Example 1 at the end of the 7-day treatment, including colon weight (CW), colon length (CL) and ulcer area.

FIGS. 4A and 4B show gross morphological differences between colons of C57BL/6 mice that were treated with TNBS to elicit colitis, and shows that treatment with the compound of Formula (I) as its malate salt (“SPH-X”) at 20 mg/kg twice daily after trinitrobenzene sulfonic acid (TNBS) exposure substantially reverses damage caused by TNBS.

FIGS. 5A and 5B show microscopic evidence of damage to colon mucosal tissues from the induced colitis model and demonstrates that treatment with 5 mg/kg or 10 mg/kg of SPH-X (the compound of Formula (I) as its malate salt) twice daily by intraperitoneal administration after exposure to TNBS treats or prevents such damage.

FIG. 6 shows that SPH-X (the compound of Formula (I) as its malate salt) significantly reduces the excess production of cytokines IL-β and IL-6 in colon mucosal tissue after exposure to TNBS.

FIG. 7 is a Western blot showing that treatment of colon tissue with TNBS results in elevated levels of TNF-α, and that treatment with SPH-X (10 mg/kg twice daily) reduces or stops formation of TNF-α.

FIG. 8A-C show the effect of Birb 796 and CFN001/01 (this identifies a specific batch of SPH-X,the compound of Formula (I) as its malate salt) on IL-6 release from human colon tissue samples of ulcerative colitis (UC) patients: FIG. 8A shows data for Donor A, FIG. 8B shows data for Donor B and FIG. 8C shows data for Donor C.

### DESCRIPTION OF SELECTED EMBODIMENTS

### General Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, malate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

### The Compound of Formula (I)

The structure of the compound of Formula (I) is shown below. The compound exhibits potent activity as a renin inhibitor and suitable pharmacokinetic characteristics for oral administration. Bioavailability in rats was about 11.5-24.5%, and in monkeys it was about 3.3-11.3%. Plasma renin activity for the compound of Formula (I) is 0.28 nM, while that for aliskiren is 0.60 nM, and activity was maintained for 24 hours even at a low dose of 0.2 mg/kg.

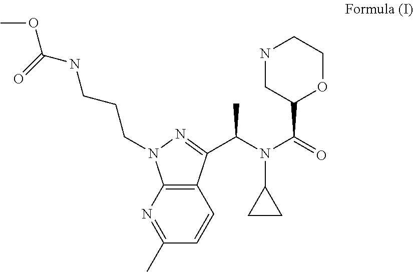

It can be formulated and administered as a neutral compound or as a pharmaceutically acceptable salt. For the experiments described herein, the compound of Formula (I) was administered as its malate salt. Synthesis and characterization of this compound are disclosed, for example, in U.S. Pat. No. 9,278,944. Preparation of the malate salt is described in U.S. Pat. No. 10,519,150. In the methods, compositions and combinations disclosed herein, the malate salt of the compound of Formula (I) is preferred.

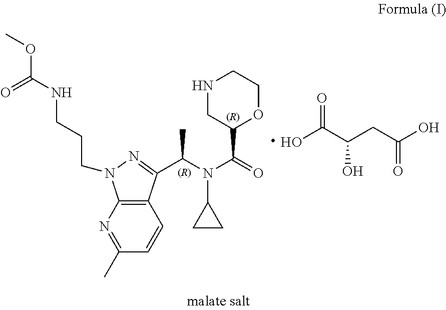

In another aspect, the present disclosure provides the compound of Formula (I), or the malate salt thereof, for use to treat an inflammatory bowel disease.

In another aspect, the invention provides a method to use a compound of Formula (I), or the malate salt thereof, for the manufacture of a medicament for the treatment of an inflammatory bowel disease.

Some aspects of the invention are summarized in the following list of enumerated embodiments.
1. A method to treat an inflammatory bowel disease in a subject in need of such treatment, which comprises administering to the subject an effective amount of a compound of Formula (I)

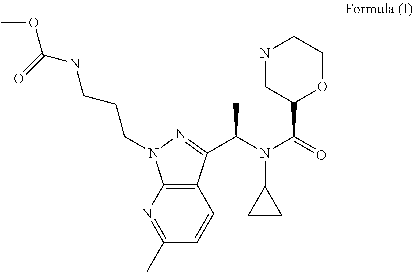

or a pharmaceutically acceptable salt thereof. In a preferred embodiment, the compound of Formula (I) is used as its malate salt
2. The method of embodiment 1, wherein the inflammatory bowel disease is ulcerative colitis.
3. The method of embodiment 1, wherein the inflammatory bowel disease is Crohn's disease.
4. The method of any one of embodiments 1-3, wherein the compound of Formula (I) or a pharmaceutically acceptable salt thereof is administered orally. In some such embodiments, the compound is administered in the form of a tablet, capsule, or softgel.
5. The method of any one of embodiments 1-3, wherein the compound of Formula (I) or a pharmaceutically acceptable salt thereof is administered as a suppository.
6. The method of any one of embodiments 1-5, wherein the compound of Formula (I) or a pharmaceutically acceptable salt thereof is administered to the subject at least once per day. In a preferred aspect of this embodiment, the compound of Formula (I) is administered twice per day.
7. The method of embodiment 6, wherein at least one dose of the compound of Formula (I) or a pharmaceutically acceptable salt thereof is administered to the subject twice daily.
8. The method any one of claims 1-7, wherein the dosage of the compound of Formula (I) or a pharmaceutically acceptable salt thereof administered to the subject is between 25 mg and 800 mg. In particular examples of this embodiment, the dosage is about 25 mg, or 50 mg, or 75 mg, or 100 mg, or 125 mg, or 150 mg, or 175 mg, or 200 mg, or 225 mg, or 250 mg, or 275 mg, or 300 mg, or 350 mg, or 400 mg, or 450 mg, or 500 mg, or 550 mg, or 600 mg, or 650 mg, or 700 mg, or 750 mg, or 800 mg.
9. The method of any one of embodiments 1-8, wherein the compound of Formula (I) or a pharmaceutically acceptable salt thereof is administered as a delayed release formulation, preferably a formulation that is configured to promote release of the compound of Formula (I) or a pharmaceutically acceptable salt thereof in the lower gastrointestinal tract, or a formulation that is configured to reduce release of the compound of Formula (I) or a pharmaceutically acceptable salt thereof in the stomach.
10. The method of any one of embodiments 1-9, wherein the subject is also treated with at least one additional IBD therapeutic. The additional IBD therapeutic can be administered admixed with the compound of Formula (I) or separately from the compound of Formula (I), and may be administered by the same or a different route of administration.
11. The method of embodiment 10, wherein the at least one additional IBD therapeutic is selected from:
   a) Anti-TNFα agents (e.g., infliximab, adalimumab, certolizumab, golimumab);
   b) Sphingosine-1-phosphate (S1P)-receptor modulators (e.g., ozanimod);
   c) Anti-adhesion (anti-integrin) agents (e.g., natalizumab, vedolizumab, ertolizumab);
   d) IL-12/IL-23 inhibitors (e.g., ustekinumab, risankizumab);
   e) Transforming growth-factor beta (TGFβinhibitors (e.g., mongersen, pirfenidone);
   f) Phosphodiesterase 4 (PDE4) inhibitors (e.g., aprimelast);
   g) Janus kinase (JAK)/signal transducers and activators of transcription (STAT) inhibitors (e.g., tofacitinib, filgotinib);
   h) Stem-cell transplants (e.g., hematopoietic stem cells, adipose-derived stem cells);
   i) Fecal microbiota transplants (FMT);
   j) Plasminogen activator inhibitor-1 (PAI-1) inhibitors (e.g., MDI-2268, tiplaxtinin);
   k) Aminosalicylates (e.g., mesalamine, balsalazide, olsalazine);
   l) Anti-inflammatory corticosteroids; and,
   m) Immune pathway inhibitors such as azathioprine, mercaptopurine, cyclosporine, and methotrexate.
12. The compound of Formula (I)

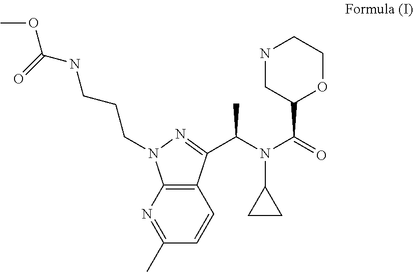

or a pharmaceutically acceptable salt thereof for use to treat an inflammatory bowel disease. In a preferred embodiment, the compound of Formula (I) is used as its malate salt
13. The compound of Formula (I) or a pharmaceutically acceptable salt thereof for use to treat an inflammatory bowel disease according to embodiment 12, wherein the inflammatory bowel disease is ulcerative colitis.
14. The compound of Formula (I) or a pharmaceutically acceptable salt thereof for use to treat an inflammatory bowel disease according to embodiment 12, wherein the inflammatory bowel disease is Crohn's disease.
15. The compound of Formula (I) or a pharmaceutically acceptable salt thereof according to any one of embodiments 12-14, wherein the compound of Formula (I) or pharmaceutically acceptable salt thereof is prepared for oral administration.
16. The compound of Formula (I) or a pharmaceutically acceptable salt thereof according to any one of embodiments 12-15, wherein the compound of Formula (I) or a pharmaceutically acceptable salt thereof is prepared to be administered to a subject at least once per week, typically at least once per day, and preferably twice per day.
17. The compound of Formula (I) or a pharmaceutically acceptable salt thereof according to embodiment 16, wherein the compound of Formula (I) or pharmaceutically acceptable salt thereof is prepared to be administered to a subject at least once daily, and preferably twice daily.
18. The compound of Formula (I) or a pharmaceutically acceptable salt thereof according to any one of embodiments 12-17, wherein the dosage of the compound of Formula (I) or a pharmaceutically acceptable salt thereof prepared for administration comprises between mg and 800 mg of the compound of Formula (I) or pharmaceutically acceptable salt thereof. In particular examples of this embodiment, the dosage is about 25 mg, or 50 mg, or 75 mg, or 100 mg, or 125 mg, or 150 mg, or 175 mg, or 200 mg, or 225 mg, or 250 mg, or 275 mg, or 300 mg, or 350 mg, or 400 mg, or 450 mg, or 500 mg, or 550 mg, or 600 mg, or 650 mg, or 700 mg, or 750 mg, or 800 mg.
19. The compound of Formula (I) or a pharmaceutically acceptable salt thereof according to any one of embodiments 12-18, wherein the compound is prepared as a delayed release formulation.
20. The compound of Formula (I) or a pharmaceutically acceptable salt thereof according to embodiment 19, wherein the delayed release formulation is configured to promote release of the compound in the lower gastrointestinal tract, or is configured to reduce release of the compound in the stomach.
21. The compound of Formula (I) or a pharmaceutically acceptable salt thereof according to any one of embodiments 12-20, wherein the compound is prepared or configured for use in combination with an additional IBD therapeutic.
22. The compound of Formula (I) or a pharmaceutically acceptable salt thereof according to embodiment 21, wherein the at least one additional IBD therapeutic is selected from:
   a) Anti-TNFα agents (e.g., infliximab, adalimumab, certolizumab, golimumab);
   b) Sphingosine-1-phosphate (S1P)-receptor modulators (e.g., ozanimod);
   c) Anti-adhesion (anti-integrin) agents (e.g., natalizumab, vedolizumab, ertolizumab);
   d) IL-12/IL-23 inhibitors (e.g., ustekinumab, risankizumab);
   e) Transforming growth-factor beta (TGFβ) inhibitors (e.g., mongersen, pirfenidone);
   f) Phosphodiesterase 4 (PDE4) inhibitors (e.g., aprimelast);
   g) Janus kinase (JAK)/signal transducers and activators of transcription (STAT) inhibitors (e.g., tofacitinib, filgotinib);
   h) Stem-cell transplants (e.g., hematopoietic stem cells, adipose-derived stem cells);
   i) Fecal microbiota transplants (FMT);
   j) Plasminogen activator inhibitor-1 (PAI-1) inhibitors (e.g., MDI-2268, tiplaxtinin);
   k) Aminosalicylates (e.g., mesalamine, balsalazide, olsalazine);
   l) Anti-inflammatory corticosteroids; and,
   m) Immune pathway inhibitors such as azathioprine, mercaptopurine, cyclosporine, and methotrexate.
23. Use of a compound of Formula (I)

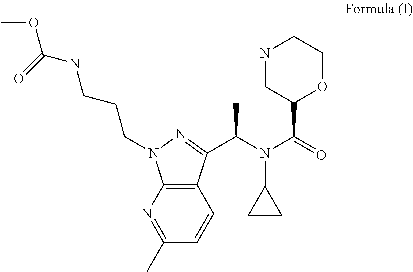

or a pharmaceutically acceptable salt thereof in the manufacture of a medicament for the treatment of an inflammatory bowel disease. In a preferred embodiment, the compound of Formula (I) is used as its malate salt
24. The use of embodiment 23, wherein the inflammatory bowel disease is ulcerative colitis.
25. The use of embodiment 23, wherein the inflammatory bowel disease is Crohn's disease.
26. The use of any one of embodiments 23-25, wherein the medicament is prepared for oral administration or as a suppository.
27. The use any one of embodiments 23-26, wherein the medicament is prepared as a dosage unit, such as a pill, capsule, tablet, or softgel containing from 25 mg to 800 mg of the compound of Formula (I) or a pharmaceutically acceptable salt thereof.
28. The use of any one of embodiments 23-27, wherein as the medicament is prepared as a delayed release formulation, preferably a formulation that promotes release of the compound of Formula (I) or a pharmaceutically acceptable salt thereof in the lower gastrointestinal tract or reduces release in the stomach.
29. The use of any one of embodiments 23-28, wherein the medicament is prepared or configured for use with at least one additional IBD therapy.
30. The use of embodiment 29, wherein the at least one additional IBD therapy is selected from:
   a) Anti-TNFα agents (e.g., infliximab, adalimumab, certolizumab, golimumab);
   b) Sphingosine-1-phosphate (S1P)-receptor modulators (e.g., ozanimod);
   c) Anti-adhesion (anti-integrin) agents (e.g., natalizumab, vedolizumab, ertolizumab);
   d) IL-12/IL-23 inhibitors (e.g., ustekinumab, risankizumab);
   e) Transforming growth-factor beta (TGFβ) inhibitors (e.g., mongersen, pirfenidone);
   f) Phosphodiesterase 4 (PDE4) inhibitors (e.g., aprimelast);
   g) Janus kinase (JAK)/signal transducers and activators of transcription (STAT) inhibitors (e.g., tofacitinib, filgotinib);
   h) Stem-cell transplants (e.g., hematopoietic stem cells, adipose-derived stem cells);
   i) Fecal microbiota transplants (FMT);
   j) Plasminogen activator inhibitor-1 (PAI-1) inhibitors (e.g., MDI-2268, tiplaxtinin);
   k) Aminosalicylates (e.g., mesalamine, balsalazide, olsalazine);
   l) Anti-inflammatory corticosteroids; and,
   m) Immune pathway inhibitors such as azathioprine, mercaptopurine, cyclosporine, and methotrexate.
31. A pharmaceutical composition comprising a compound of Formula (I)

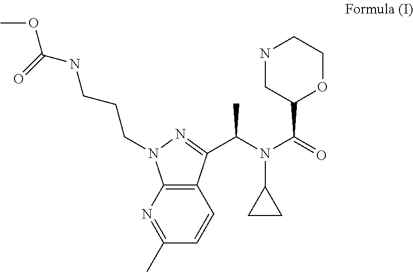

or a pharmaceutically acceptable salt thereof admixed with an additional IBD therapeutic agent. In preferred embodiments, the compound of Formula (I) is used as its malate salt
32. The pharmaceutical composition of embodiment 31, which is a solid dosage form for oral administration, a softgel, or a suppository.
33. The pharmaceutical composition of embodiment 31 or 32, which comprises between 25 mg and 800 mg of the compound of Formula (I) or a pharmaceutically acceptable salt thereof.
34. The pharmaceutical composition according to any one of embodiments 31-33, wherein the compound of Formula (I) or a pharmaceutically acceptable salt thereof is prepared as a delayed release formulation.
35. The pharmaceutical composition according to any one of embodiments 31-34, wherein the pharmaceutical composition is configured to promote release of the compound of Formula (I) or a pharmaceutically acceptable salt thereof in the lower gastrointestinal tract, or is configured to reduce release of the compound of Formula (I) or a pharmaceutically acceptable salt thereof in the stomach.
36. The pharmaceutical composition according to any one of embodiments 31-35, wherein the at least one additional IBD therapeutic is selected from:
   a) Sphingosine-1-phosphate (S1P)-receptor modulators (e.g., ozanimod);
   b) Transforming growth-factor beta (TGFβ) inhibitors (e.g., mongersen, pirfenidone);
   c) Phosphodiesterase 4 (PDE4) inhibitors (e.g., aprimelast);
   d) Janus kinase (JAK)/signal transducers and activators of transcription (STAT) inhibitors (e.g., tofacitinib, filgotinib);
   e) Plasminogen activator inhibitor-1 (PAI-1) inhibitors (e.g., MDI-2268, tiplaxtinin);
   f) Aminosalicylates (e.g., mesalamine, balsalazide, olsalazine);
   g) Anti-inflammatory corticosteroids; and,
   h) Immune pathway inhibitors (e.g., azathioprine, mercaptopurine, cyclosporine, methotrexate, TNF-α inhibitors).

In any of the foregoing embodiments, the compound of Formula (I) can be used or administered as a malate salt.

### Pharmaceutical Compositions, Combinations, and Other Related Uses

In still another aspect, the present disclosure provides a pharmaceutical composition comprising a compound of Formula (I) or a pharmaceutically acceptable salt thereof admixed with at least one pharmaceutically acceptable carrier or excipient, wherein the composition is configured for use to treat an IBD. In some embodiments, the composition further comprises an additional therapeutic agent useful for treating an IBD. In some embodiments, the pharmaceutical composition is adapted to delay release of the compound of Formula (I) or a pharmaceutically acceptable salt thereof, in particular to promote release of the compound of Formula (I) or a pharmaceutically acceptable salt thereof primarily in the lower gastrointestinal tract and/or to reduce release of the compound of Formula (I) or a pharmaceutically acceptable salt thereof in the stomach.

In yet another aspect, the present disclosure provides for the compound of Formula (I) or a pharmaceutically acceptable salt thereof for use to treat an inflammatory bowel disease. the compound can be used as its malate salt.

In yet another aspect, the present disclosure provides for the use of the compound of Formula (I) or a pharmaceutically acceptable salt thereof for the manufacture of a medicament for treating an inflammatory bowel disease. In some of these embodiments, the malate salt of the compound of Formula (I) is used.

### Formulations

Any suitable formulation of the compound of Formula (I) or a pharmaceutically acceptable salt thereof or combinations comprising the compound of Formula (I) or a pharmaceutically acceptable salt thereof can be prepared. See generally, Remington's Pharmaceutical Sciences, (2000) Hoover, J. E. editor, 20th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Preferably, the compound of Formula (I) or a pharmaceutically acceptable salt thereof is formulated for oral administration, typically as a tablet or capsule. In some embodiments the malate salt of the compound of Formula (I) is used.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water-soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

### Delayed Release Formulations

A compound of Formula (I) can be formulated for immediate release and quick absorption, or it can be formulated for delayed release. In some embodiments, the compound is formulated for delayed release, using methods and compositions that promote delivery of the active ingredient in the lower gastrointestinal tract, after the administered formulation has passed through the stomach. Such methods include known enteric coatings that slow or prevent release of the compound of Formula (I) or a pharmaceutically acceptable salt thereof in the stomach, so that the active drug is primarily released in the intestines, to enhance direct delivery to the tissues most affected by IBD. Some useful methods for delayed release formulations are described for example in B. Singh, Modified-release solid formulations for Colonic Delivery, Recent Patents on Drug Delivery and Formulations 2007, Vol. 1(1), 53-63. The compound of Formula (I) or a pharmaceutically acceptable salt thereof can be formulated using such methods to reduce dissolution in the stomach, and/or to increase dissolution and absorption in the lower gastrointestinal (GI) tract, in order to increase availability of the active drug in the targeted tissues.

Methods to achieve delayed release can utilize a single or a combination of two or more of the following: pH-controlled (or delayed-release) systems, time-controlled (or time-dependent) systems, microbially-controlled systems, and pressure-controlled systems

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of a compound of Formula (I) or a pharmaceutically acceptable salt thereof to a subject in need of treatment for an inflammatory bowel disease. The compound of Formula (I) can be administered as a neutral compound, or it can be administered as a pharmaceutically acceptable salt. In some embodiments, it is administered as a malate salt. The compound of Formula (I) or a pharmaceutically acceptable salt thereof can be administered as a single agent, or it may be combined with an additional therapeutic agent. Optionally, the compound of Formula (I) or a pharmaceutically acceptable salt thereof may be administered in combination with one or more additional therapeutic agents, particularly therapeutic agents known to be useful for treating an inflammatory bowel disease. These include but are not limited to:
a) Anti-TNFα agents (e.g., infliximab, adalimumab, certolizumab, golimumab);
b) Sphingosine-1-phosphate (S1P)-receptor modulators (e.g., ozanimod);
c) Anti-adhesion (anti-integrin) agents (e.g., natalizumab, vedolizumab, ertolizumab);
d) IL-12/IL-23 inhibitors (e.g., ustekinumab, risankizumab);
e) Transforming growth-factor beta (TGFβ) inhibitors (e.g., mongersen, pirfenidone);
f) Phosphodiesterase 4 (PDE4) inhibitors (e.g., aprimelast);
g) Janus kinase (JAK)/signal transducers and activators of transcription (STAT) inhibitors (e.g., tofacitinib, filgotinib);
h) Stem-cell transplants (e.g., hematopoietic stem cells, adipose-derived stem cells);
i) Fecal microbiota transplants (FMT);
j) Plasminogen activator inhibitor-1 (PAI-1) inhibitors (e.g., MDI-2268, tiplaxtinin);
k) Aminosalicylates (e.g., mesalamine, balsalazide, olsalazine);
l) Anti-inflammatory corticosteroids; and,
m) Immune pathway inhibitors such as azathioprine, mercaptopurine, cyclosporine, and methotrexate.

The methods further include use of the compound of Formula (I) or a pharmaceutically acceptable salt thereof in combination with other therapies for treating IBD, including therapeutic methods such as fecal microbiota transplants and stem cell transplants.

Use of the compound of Formula (I) or a pharmaceutically acceptable salt thereof in combination with another IBD therapeutic agent or therapy includes co-administration of the compound of Formula (I) or a pharmaceutically acceptable salt thereof with another IBD therapeutic agent as well as concurrent use of another IBD therapeutic agent or therapy in a given patient where the other IBD therapeutic agent or therapy is administered separately from the compound of Formula (I) or a pharmaceutically acceptable salt thereof, even on different days from administration of the compound of Formula (I), provided that the different therapeutic treatments are administered in a sequence and time window where both are expected to provide therapeutic benefits to the subject concurrently. Thus, the compound of Formula (I) or a pharmaceutically acceptable salt thereof is used in combination with an IBD therapeutic agent or therapy whenever the subject is expected to receive IBD treatment therapeutic effects from both the compound of Formula (I) and the other IBD therapeutic agent or therapy over any period of time.

The additional IBD therapeutic agent may be administered in a separate pharmaceutical composition from the compound of Formula (I) or a pharmaceutically acceptable salt thereof, or it may be included with the compound of Formula (I) or a pharmaceutically acceptable salt thereof when their route of administration and timing of administration are compatible for inclusion in a single pharmaceutical composition. The additional IBD therapeutic agent may be administered simultaneously with, prior to, or after administration of the compound of Formula (I) or a pharmaceutically acceptable salt thereof.

### Methods of Using Compounds of Formula (I) and Pharmaceutical Compositions Thereof

Selection of a route of administration and a suitable formulation for administering the compound of Formula (I) or a pharmaceutically acceptable salt thereof is within the ordinary skill of a physician in view of information available in the art about the pharmacokinetic properties and chemical properties of the compound of Formula (I) in combination with information provided herein. The physician would be able to monitor effectiveness of such treatments and adjust dosage and frequency of administration using known methods.

To practice the method of the present invention, the compound of Formula (I) or a pharmaceutically acceptable salt thereof, and pharmaceutical compositions thereof, may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

In a particular embodiment of the methods of the invention, a compound of Formula (I) or a pharmaceutically acceptable salt thereof is administered orally. A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch.

When aqueous suspensions or emulsions of a compound of Formula (I) or a pharmaceutically acceptable salt thereof are administered orally, the compound can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In preferred embodiments, the compound of Formula (I) or a pharmaceutically acceptable salt thereof is administered orally in the form of a tablet, capsule, softgel, or suppository, typically comprising 25 mg to 800 mg of the compound (or of the malate salt of the compound) per dose. A single dose may be contained in a single dosage form such as a pill or capsule, or a single dose may require use of two, three, four, or more single dosage forms such as pills or capsules. In some embodiments, a single dosage form such as a pill, tablet or capsule contains an appropriate amount of the compound of Formula (I) or its malate salt for a single dose, e.g., about 25 mg, or 50 mg, or 75 mg, or 100 mg, or 125 mg, or 150 mg, or 175 mg, or 200 mg, or 225 mg, or 250 mg, or 275 mg, or 300 mg, or 350 mg, or 400 mg, or 450 mg, or 500 mg, or 550 mg, or 600 mg, or 650 mg, or 700 mg, or 750 mg, or 800 mg. In some embodiments, a single pill, tablet, softgel, suppository, or capsule containing the desired dose for an adult is administered at least once per day to a subject in need of treatment for an IBD. In a preferred embodiment, a dosage comprising the compound of Formula (I) is administered twice daily.

In addition, the compound of Formula (I) or a pharmaceutically acceptable salt thereof may be administered alone or in combination with other therapeutic agents, as disclosed herein. Combination therapies according to the present invention comprise the administration of at least one dosage of the compound of Formula (I) or a pharmaceutically acceptable salt thereof and at least one other pharmaceutically active ingredient useful for the treatment of IBD. The dosage of the compound of Formula (I) or a pharmaceutically acceptable salt thereof and other pharmaceutically active agents may be administered separately or together. The amounts of the compound of Formula (I) or a pharmaceutically acceptable salt thereof and other pharmaceutically active agent(s) and the relative timings of administration will be selected in order to achieve the desired combined therapeutic effect.

The following Examples are provided to illustrate the biological activity of the compound of Formula (I) thereof in a colitis model system, and are not intended to limit the scope of the invention.

### EXAMPLES

### Example 1. Effect of Formula (I) Malate Salt on Induced Colitis

Colitis was induced in Wistar rats by intracolonic administration of DNBS. Rats were sorted into six groups as described below. The first group was DNBS-naive, while groups 2-6 were each treated with DNBS on day one only. The second group, treated with DNBS, and no therapeutic agent, served as a diseased control. The third group was treated with tofacitinib, a known treatment for ulcerative colitis, as a positive therapeutic comparator. The fourth and fifth groups were treated with different doses of the malate salt of the compound of Formula (I), and the sixth group was treated with a combination of a malate salt of the compound of Formula (I) and tofacitinib. Animals were treated daily as described below, starting shortly after DNBS was administered. Treatment continued for 7 days, during which time stool consistency was monitored. After 7 days, the animals were euthanized and the colon of each animal was evaluated for weight, length and area of ulceration.

The test methods and results are summarized below.

### Animals

| | |
|---|---|
| Animal species and strain: | Wistar rats |
| History of treatment: | Naive |
| Sex, age and weight: | Male, 5-6 weeks, 140-160 g |
| Breeder/supplier: | Shanghai SLAC Laboratory Animal Co. |
| | Ltd. |
| Test Facility: | PharmaLegacy Laboratories Vivarium |
| Adaptation: | Not less than 7 days |
| Room: | SPF Room |
| Room temperature: | 19-26° C. |
| Room relative humidity: | 40-70% |
| Light cycle: | Fluorescent light for 12-hour light |
| | (08:00-20:00) and 12-hour dark |
| Animal hosting: | 2-3 rats/cage by treatment group |
| Food: | Free access to food (irradiated, Shanghai |
| | SLAC Laboratory Animal Co. Ltd., China) |
| Water: | Free access to water (municipal tap water |
| | filtered by water purification system) |

A total of 82 male Wistar rats were obtained from Shanghai SLAC Laboratory Animal Co. Ltd. The animals were specific pathogen free and approximately 4-5 weeks old upon arrival.

### Allocation to Treatment Groups

Animals were assigned to treatment groups by randomization using BioBook software to achieve similar group mean weights on Day-1, which provided for control of bias.

TABLE 1
Treatment Groups

| | | Model | | | Conc. | Dosage | | Dosing |
|---|---|---|---|---|---|---|---|---|
| Group | Test Article | Induction | N | Route | mg/mL | mL/kg | mg/kg | frequency |
| 1 | Naive (ethanol only | Ethanol | 12 | p.o. | N/A | 10 | N/A | q.d. Day 1-7 |
| | control)ª | | | | | | | |
| 2 | Vehicle (DNBS | DNBS | 14 | p.o. | N/A | 10 | N/A | q.d. Day 1-7 |
| | control) ª | | | | | | | |
| 3 | Tofacitinib^{b} | DNBS | 14 | p.o. | 3 | 10 | 30 | b.i.d. Day 1-7 |
| 4 | Formula I malate | DNBS | 14 | p.o. | 3 | 10 | 30 | q.d. Day 1-7 |
| | salt low dosageª | | | | | | | |
| 5 | Formula I malate | DNBS | 14 | p.o. | 10 | 10 | 100 | q.d. Day 1-7 |
| | salt high dosageª | | | | | | | |
| 6 | Formula I malate | DNBS | 14 | p.o. | 10 | 10 | 100 | q.d. Day 1-7 |
| | salt high dosage^{a} | | | | | | | |
| | Tofacitinib ^{b} | | | | 3 | 10 | 30 | b.i.d. Day 1-7 |

^{a}vehicle for test articles was distilled water.
^{b}vehicle was 0.5% CMC-Na

Colitis was induced in Wistar rats by intracolonic administration of 0.5 mL DNBS solution (50 mg/mL DNBS in 30% ethanol) in Groups 2-6 on day 1. At the same time Group 1 received 30% ethanol (0.5 mL) intracolonically as ethanol control.

A total of 82 male Wistar rats were randomly assigned to 6 groups, as follows:
GROUP 1: NAIVE (ETHANOL ONLY CONTROL), N=12
GROUP 2: VEHICLE (DNBS CONTROL), N=14
GROUP 3: TOFACITINIB, 30 MPK, P.O., BID, N=14
GROUP 4: FORMULA I MALATE SALT, 30 MPK, P.O., QD, N=14
GROUP 5: FORMULA I MALATE SALT, 100 MPK, P.O., QD, N=14
GROUP 6: FORMULA I MALATE SALT (100 MPK, QD)+TOFACITINIB (30 MPK, BID), P.O., N=14

Body weight and stool consistency were recorded daily for all of the subject animals. The animals were sacrificed on day 7. Each colon was collected. Ulcer area, distal colon weight, colon length, and photos of the relevant colon areas were recorded. Colon tissues were split longitudinally into three pieces and one piece of colon was immediately fixed in 10% neutral buffered formalin. The other two pieces of colon were collected and snap-frozen in liquid nitrogen and stored at −80° C.

### Abbreviations

DNB S: 2, 4-Dinitrobenzenesulfonic acid,

IBD: Inflammatory Bowel Disease

CMC-Na: Sodium Carboxymethylcellulose,

The test articles were prepared as follows: Formula (I) malate salt was weighed by electronic balance and dissolved in distilled water and then vortexed completely to dissolve it.

As a comparator compound, Tofacitinib was included in the testing of Formula I malate salt. Tofacitinib is approved for treating rheumatoid arithritis, and for treating moderate to severe ulcerative colitis.

| | |
|---|---|
| Reference compound: | Tofacitinib |
| Supplier: | PharmaBlock Sciences |
| Storage conditions: | 2~8° C. |
| Cat No .: | PBN2011586 |
| Lot No .: | PB0000461-169-01 |

A 3 mg/mL Tofacitinib suspension was prepared in 0.5% sodium carboxymethyl cellulose: a fresh sample was prepared twice each week to ensure quality.

DNBS was dissolved in 30% ethanol at a concentration of 50 mg/mL.

Reference drug solution: Tofacitinib was diluted in 0.5% CMC-Na to the concentration of 3 mg/mL.

### Induction of Colitis

On Day −1, animals were randomized into 6 groups (see treatment groups table 1), and were food-fasted for 40 hours. For energy intake, 5% glucose in saline (10 mL/kg, s.c.) was supplied during fasting.

On Day 1 of the study, the fasting animals were anesthetized with Zoletil (i.p., 25 mg/kg), Zolazepam (i.p., 25 mg/kg) and Xylazine (i.p., 5 mg/kg).

For Group 2-6, colitis was induced by intracolonic administration of 0.5 mL DNBS using a catheter which was inserted into the colon via the anus up to the splenic flexure (8 cm from the anus). Group 1 received 30% ethanol, also via intracolonic administration. Animals exposed to DNBS or ethanol were held head down for 15 min and then kept in a Trendelenburg position until they revived in order to avoid reflux.

### Treatment

Group 1: animals were administered orally with distilled water 4 hours after 30% ethanol from day 1 till day 7, q.d.

Group 2: animals were administered orally with distilled water 4 hours after 30% ethanol from day 1 till day 7, q.d.

Group 3: animals were administered orally with 30 mg/kg (mpk) Tofacitinib 4 hours after DNBS from day 1 till day 7, b.i.d.

Group 4-5: animals were administered orally with different dosages of Formula I malate salt 4 hours after DNBS from day 1 till day 7, q.d.

Group 6: animals were administered orally with 100 mpk of the malate salt of the compound of Formula (I) (referred to herein as Formula I malate salt) q.d. and 30 mpk Tofacitinib, b.i.d. 4 hours after DNBS from Day 1 till day 7.

### Assessment of Colitis

### Body Weight

Body weights were recorded daily throughout the study. The percent weight change on each day in relation to the starting weight was calculated using the formula:

[(Weight on day X−Initial weight)/Initial weight]×100

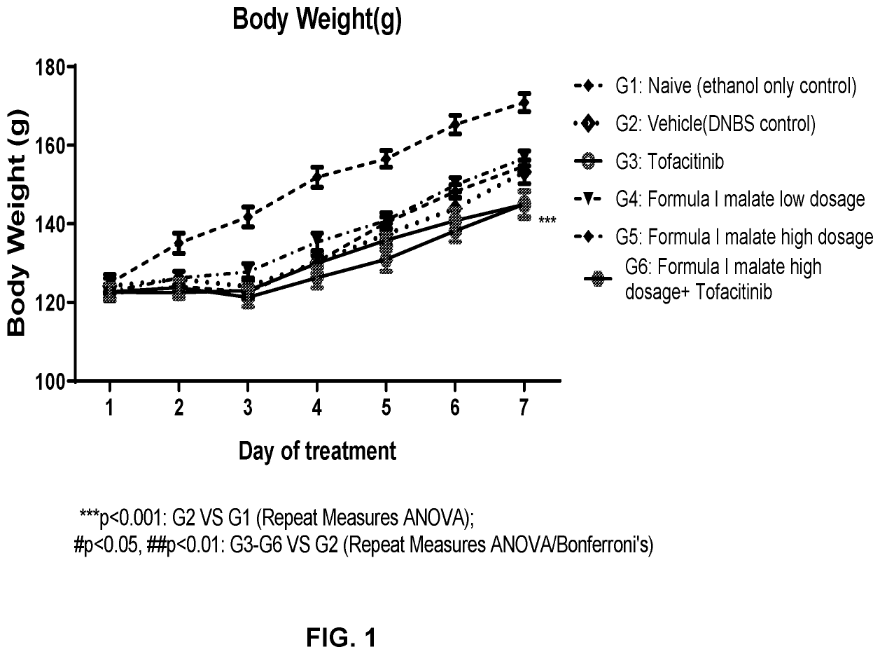

Body weights of test animals over the course of treatment are summarized in FIG. 1.

### Score for Stool Consistency

During the experiment, stool was monitored daily and scored consistency (0=formed, 1=moist/sticky, 2=loose, 3=liquid) as an indicator of colitis severity.

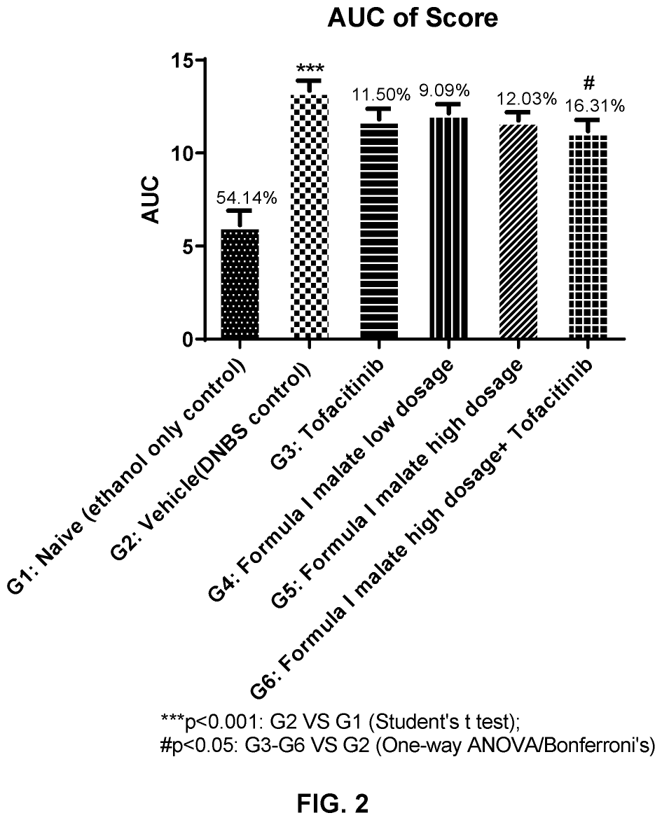

Stool consistency scoring for the animals over the course of treatment was graphed for the 7-day experiment using the above scoring, and the graph was used to calculate the area under the curve (AUC) for each treatment group. The AUC for each treatment and control group is shown in FIG. 2.

### Colon Weight and Length and Ulcer Area

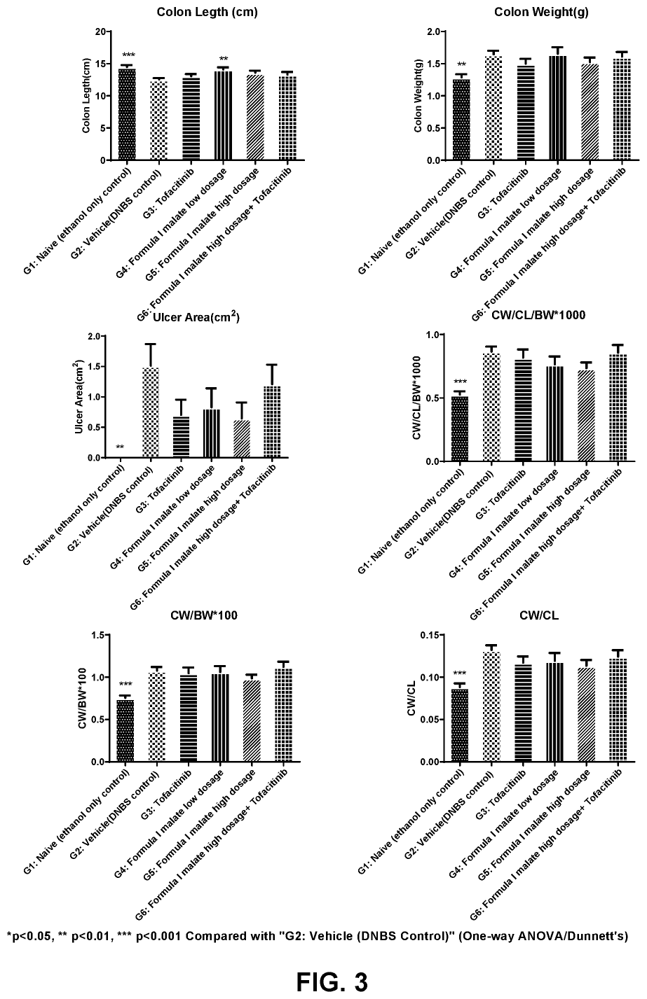

On Day 7, all animals were sacrificed by CO₂ asphyxiation followed by cervical dislocation. The abdomen was opened by a midline incision. The colon was emptied of its content, rinsed and weighed. The length of the colon (from cecum end to the anus) and the ulcerated surface area of the colon interior were measured. Macroscopic evaluations of colon length (CL), colon weight (CW), and extent of ulceration (area) were measured for all treatment and control groups, and those results along with CW/CL, CW/BW (body weight), and CW/CL/BW are shown in FIG. 3.

Note: If the shape of ulceration is irregular, ulcerated segments were pieced together to form a rectangle and then the area of the rectangle was measured (The area=length*width).

### Sample Collection

After evaluation of colon length and weight, longitudinal tri-section of the entire colon was done, and two pieces of colon were snap-frozen in liquid nitrogen and stored at −80° C. Another piece was fixed in 10% neutral buffered formalin for histopathology evaluation.

### Clinical Observations

Animals were observed daily for signs of illness and general reaction to surgery and to treatments. All exceptions to normal healthy appearance and behavior were recorded and detailed in standard PharmaLegacy Laboratories clinical observations forms.

### Statistics

Group means±S.E.M. were calculated for body weight, colon length, colon weight, colon weight/length, colon weight/body weight, ulcer area and other pending parameters. Statistical analyses were performed using Graphpad Prism, SPSS or Sigmaplot. The specific statistical tests used are identified in the Figure legends. A value of p<0.05 was considered statistically significant.

### Results

Significantly decreased body weight, increased stool consistency score and AUC of stool consistency score, decreased colon length, increased colon weight, and increased ulcer area were observed for all of the groups that were treated with DNBS when compared to the control, treated only with vehicle (ethanol). This demonstrates that the model system induced colitis symptoms. The ratios CW/CL, CW/BW and CW/CL/BW were also higher in the treatment groups compared to the DNBS-naive group.

Tofacitinib was included as a positive control expected to reduce colitis effects but acting via a different mechanism than Formula I malate salt. In the Tofacitinib treatment group, receiving 30 mg/kg BID, the CW/CL, CW/BW and CW/CL/BW ratios improved by 37%, 9% and 14%, respectively.

Animals in the treatment group receiving Formula I malate salt at 30 mg/kg q.d. exhibited significantly increased colon length. The CW/CL, CW/BW and CW/CL/BW inhibition ratios improved by 30%, 6% and 29%, respectively relative to the DNBS-treated control group.

In animals in the group treated with Formula I malate salt at 100 mg/kg per day, the CW/CL, CW/BW and CW/CL/BW ratios improved by 44%, 29% and 39%, respectively. This demonstrates that the compound of Formula I reduced the extent and/or severity of lesions caused by colitis at both dosages, and at the higher dose, Formula I malate salt appears to be more effective than the comparator, tofacitinib, for treating induced colitis.

Formula I malate salt combined with tofacitinib significantly decreased the AUC of stool consistency score. This suggests that a combination of Formula I malate salt and tofacitinib might be advantageous for treating IBD.

### Example 2. Effect of Formula (I) Malate Salt on TNBS-Induced Colitis in C57BL/6 Mouse

Colitis was induced in C57BL/6 mice by instillation of trinitrobenzene sulfonic acid (TNBS) in the colons according to conventional methods. See Antoniou, et al., Ann. Medicine and Surgery, vol. 11, 9-15 (2016). The mice were then treated with 5-20 mg/kg of the Formula (I) malate salt (“SPH-X”) or PBS (control) as indicated for each study.

### Macroscopic Observations

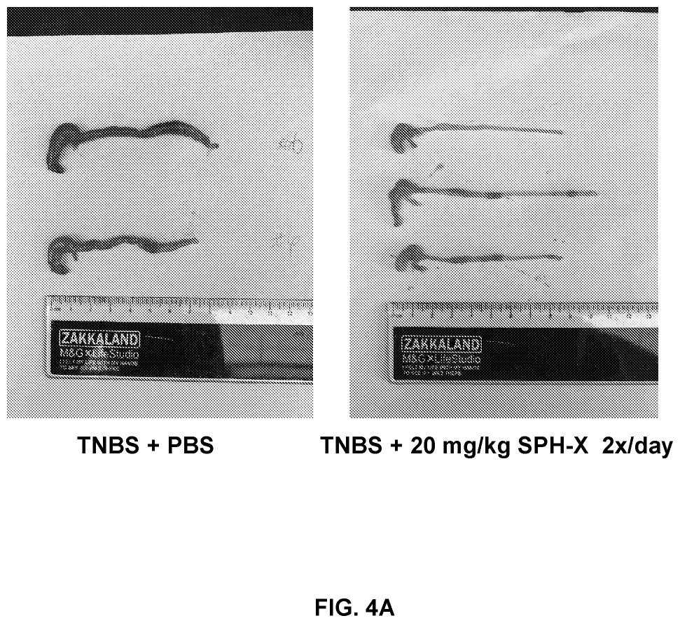

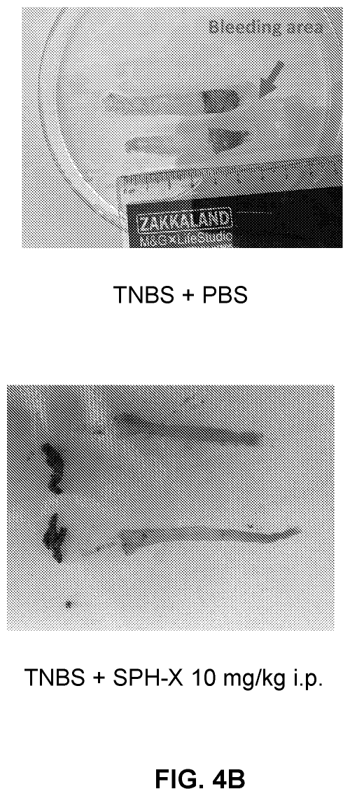

FIG. 4A shows gross morphology of colons of mice 7 days after instillation of TNBS, to compare with the colons of mice treated twice daily with 20 mg/kg SPH-X by intraperitoneal delivery to ones that received vehicle (PBS) instead. Colons from the vehicle (PBS)-treated mice are shorter and swollen, and do not show distinct fecal pellet formation; this is as expected for the ulcerative colitis model. Colons from mice treated with SPH-X (20 mg/kg BID) appear more normal; they are longer and thinner than the colons of vehicle-treated mice and exhibit distinct fecal pellets. This shows that the SPH-X treatment treats or prevents the injury that TNBS would otherwise cause at a gross physical level in the colitis model.

FIG. 4B shows TNBS-treated colons dissected longitudinally, to expose the interior of the colons. Colons treated only with TNBS as described above exhibit bleeding at the distal ends, while colons of animals that received 10 mg/kg SPH-X intraperitoneally after instillation of TNBS do not show such damage. Both external and internal macroscopic observations show that TNBS treatment causes clear, gross morphological injuries consistent with colitis, and those injuries are substantially prevented or reversed by intraperitoneal treatment with SPH-X (Formula (I) malate salt).

### Microscopic Observations

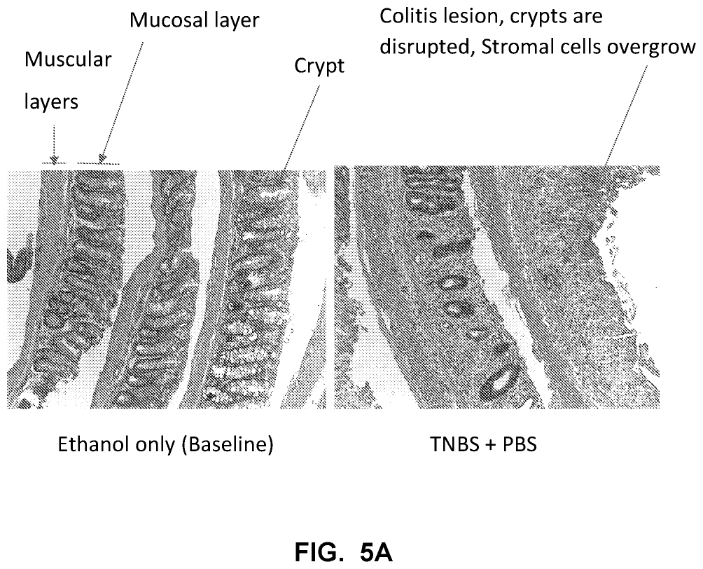

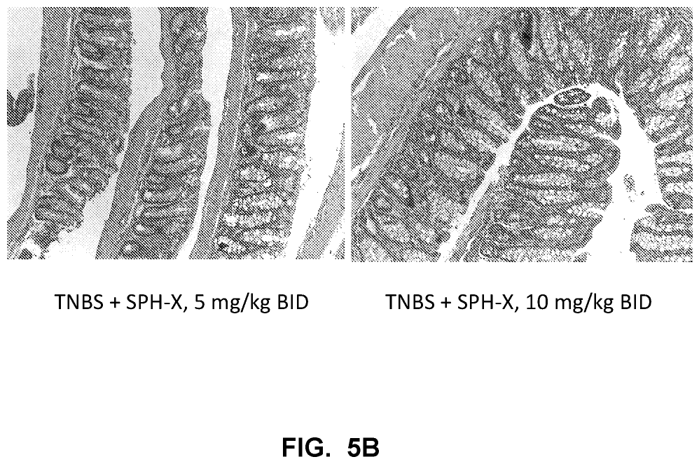

FIGS. 5A-B show histological observations of colon sections from C57BL/6 mice. All animals were sacrificed on day 3 after the TNBS instillation to induce colitis as described above, and tissues are visualized by H&E (hematoxylin and eosin) staining.

The first panel in FIG. 5A shows tissue from a colon instilled with ethanol only (no TNBS), which serves as a baseline. The second panel in FIG. 5A shows a colon instilled with TNBS followed by twice daily intraperitoneal treatment with PBS (colitis control), which exhibits tissue injury typical for colitis injury caused by TNBS.

FIG. 5B shows the effect of SPH-X at 5 or 10 mg/kg BID in TNBS-instilled mice. The first panel in FIG. 5B is tissue of a colon instilled with TNBS and treated with 5 mg/kg SPH-X BID; it shows that this dosage of SPH-X substantially prevents or treats any injury caused by TNBS instillation. The second panel in FIG. 5B is tissue from a colon instilled with TNBS and treated with 10 mg/kg SPH-X twice daily, administered intraperitoneally. It, too, shows that treatment with SPH-X substantially protects the colon from injury caused by TNBS. These images show that SPH-X treats or prevents injury caused by TNBS in the mouse colitis model at a microscopic level.

### Biochemical Observations

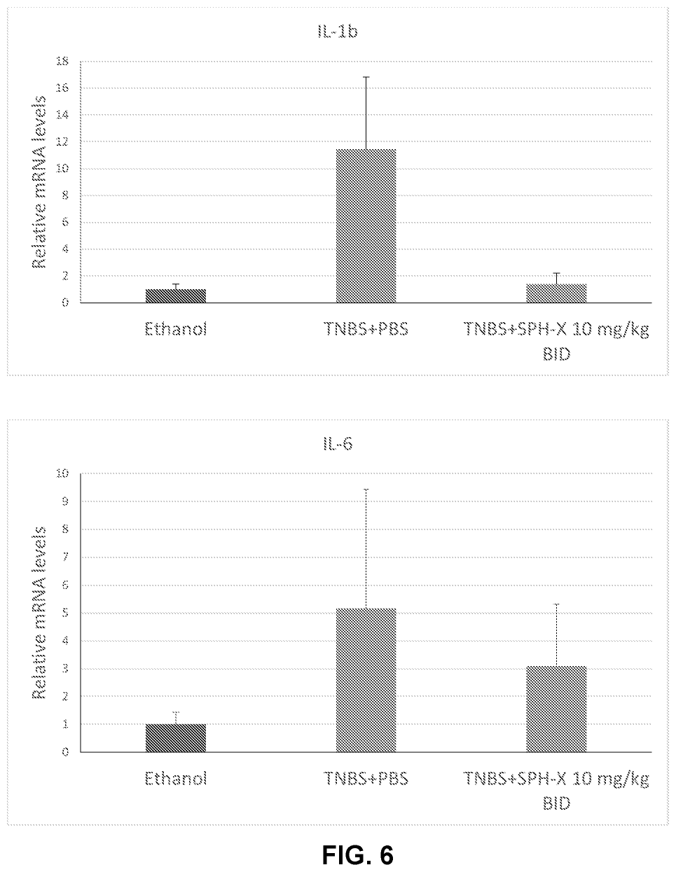

To assess the effect of SPH-X in this model at the molecular level, colitis was again induced in mice by TNBS instillation (three per treatment group), using ethanol as a control. Test animals were treated with either vehicle (PBS) or SPH-X (10 mg/kg i.p., twice daily) for three days following the TNBS injury and were then sacrificed. Colonic mucosa were isolated from each test animal and used to prepare mRNA. The mRNA was reverse transcribed to provide cDNAs. Cytokines IL-1(3 and IL-6 were quantified by qRT-PCR. Table 2 shows the data from qRT-PCR, normalized to the ethanol control (no TNBS treatment), and the results are shown graphically in FIG. 6.

TABLE 2

| | | | TNBS + SPH-X |
|---|---|---|---|
| | Ethanol | TNBS + PBS | 10 mg/kg BID |
| IL-1b | 1 | 11.44484 | 1.399821 |
| IL-6 | 1 | 5.164949 | 3.093363 |
| SEM | | | |
| | 0.412269 | 5.378737 | 0.816367 |
| | 0.437348 | 4.274689 | 2.231583 |

TNBS instillation caused significant elevation of both cytokines IL-1β and IL-6 relative to the ethanol control, as expected for the colitis model. Samples from the SPH-X mice showed a significant reduction of levels of these cytokines. Since the colitis injury is believed to be mediated by these (and likely other) cytokines, this demonstrates that SPH-X reduces the tissue injury caused by TNBS at a biochemical level and may act at least in part by reducing cytokine release.

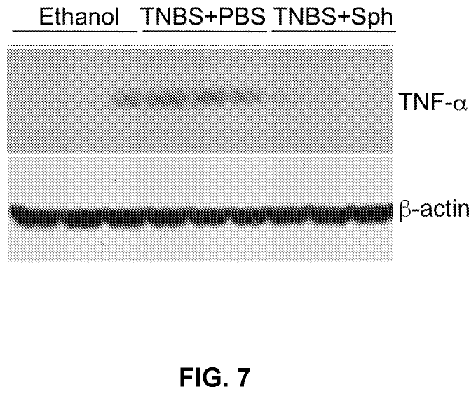

Finally, mucosal lysates from test animals treated as described for the cytokine analysis were prepared and analyzed by Western blot to see how SPH-X affects TNF-α protein levels in the colitis model. FIG. 7 shows the results of the analysis; each lane represents one mouse and beta-actin was included as a control. The ethanol control animals showed no detectable TNF-aα protein. In contrast, the TNBS colitis model animals exhibited readily detectable levels of TNF-α. Treatment with 10 mg/kg SPH-X (twice daily for three days; labeled as Sph in the figure) reversed this effect of TNBS installation, as no TNF-α protein was seen in mucosal isolates from mice treated with SPH-X.

### Example 3. Effect of Formula (I) Malate Salt (CFN001/01) on Human Ulcerative Colitis and Normal Mucosal Colon Tissue

Human colon tissues from healthy donors and from donors with ulcerative colitis (UC) were tested under conditions designed to elicit cytokine production to determine whether the compound of Formula (I) reduces cytokine production in tissue from donors with ulcerative colitis.

Human UC and normal gastrointestinal tissue were obtained from surgical residual sources of six donors, three normal and three with ulcerative colitis, with informed consent. Donors were pre-screened to exclude subjects who had received any anti-cytokine therapeutics within the past month. Smooth muscle was separated from the mucosa and attached submucosa for each sample. A scalpel was used to dissect each sample to produce 18 full thickness mucosal biopsies, approximately 5 mm x 5 mm in size. The samples were washed and held in culture medium for about 10 minutes while culture plates were prepared.

### Preparation of Test Articles

Staphylococcus aureus enterotoxin B (SEB) (100 μg/mL stock solution) was prepared in phosphate-buffered saline (PBS). The 100 μg/mL stock solution was then diluted in PBS to a concentration of 10 μg/mL, so that adding 50 μL of this solution to 9.95 mL of the culture medium yielded a final well concentration of 50 ng/mL SEB. SEB was added to provide a consistent baseline level of cytokine production.

Birb 796 (positive control, Selleck Chemicals catalogue No: S1574) was purchased as a powder. Birb 796 is a broad-spectrum inhibitor of p38 MAP kinase known to inhibit cytokine formation. A 10 mM stock solution was prepared in DMSO. This solution was then added to culture medium at a volume of 1μL per 10 mL of medium to achieve the appropriate concentration of 1μM Birb796 and a DMSO concentration of 0.01%.

CFN001/01 (Formula (I) malate salt) was provided as a powder, and a stock solution of 10 mM was prepared in distilled water and stored at −20° C. A fresh 10 mM aliquot was used on each experimental day. Working solutions were prepared by diluting the 10 mM aliquot in distilled water to concentrations of 300, 100, 30 and 10 μM.

Each working concentration was then added to media at 1 μl per 1 mL of media to yield final concentrations of 300, 100, 30 and 10 nM in the wells.

CMRL culture media was prepared by standard methods. Vehicle was distilled water and was added to culture media at 1 μl per 1 mL to match the test compound for the control.

Samples were placed apical (mucosal) side facing upwards on a Netwell filter at the liquid-air interface. The biopsy samples were then incubated at approximately 37° C. in a high O₂ atmosphere in culture medium fortified with the appropriate control or test article, Formula I malate salt (“CFN001/01”). The p38 MAP kinase inhibitor Birb 796 (CAS 285983-48-4) was used as a positive control. Each test condition was evaluated in triplicate biopsies per donor. The test medium for each sample also contained 50 ng/mL Staphylococcus aureus enterotoxin B (SEB) to provide a consistent baseline level of cytokine production. Each test condition was evaluated in triplicate biopsies per donor.

### Test Conditions

1. Vehicle control

2. BIRB 796-1μM

3. CFN001/01-300 nM

4. CFN001/01-100 nM

5. CFN001/01-30nM

6. CFN001/01-10 nM

At approximately 18-hours post-culture start, media samples were collected and snap frozen in liquid nitrogen and stored at approximately −80° C. until they were prepared for ELISA analysis.

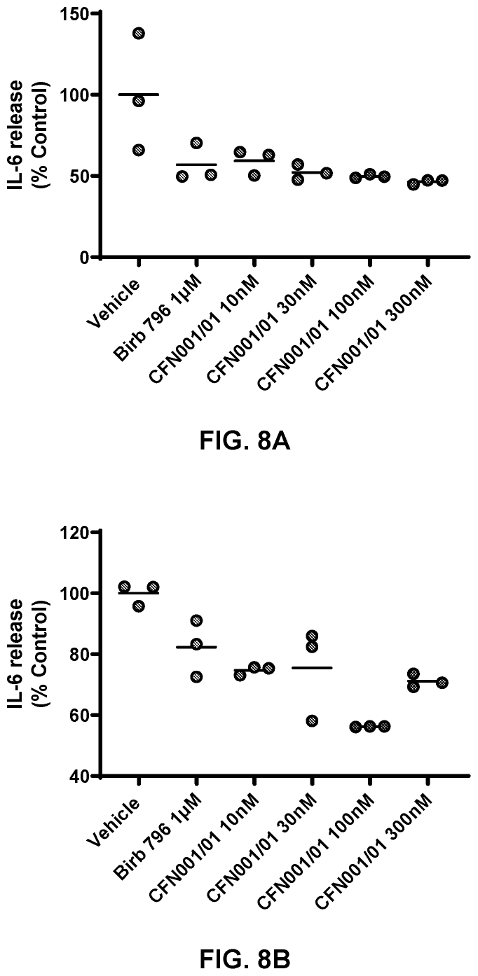

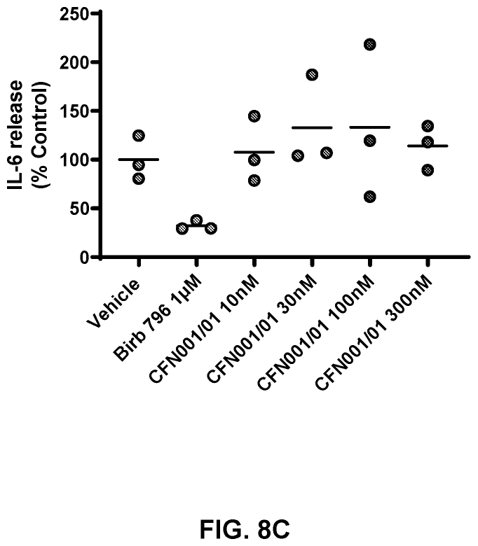

Culture media samples were then analyzed for IL-1β, IL-17A, TNF-α, IL-6, and IL-23 using multiplex ELISA. The multiplex ELISA platform used was the Luminex Magpix® system using Luminex xMAP® compatible magnetic bead technology. Each analyte was quantified by interpolation against a standard curve generated on the same 96 well analysis plate. Each sample was analyzed in duplicate with the mean value being used for the graphs in FIG. 8.

Selected ELISA results are presented in graphical format in FIG. 8, where each graph represents data for one UC subject; the graphs show the effect of CFN001/01 on IL-6 release by mucosal tissue samples. Each graph in FIG. 8 summarizes results from a single UC subject, and each dot represents cytokine release from 1 of 3 replicate tissue samples. The horizontal solid line for each test condition represents the mean of the 3 individual donor mean values for that test condition, expressed as a percentage of the vehicle control.

As the graphs in FIG. 8A-C show, CFN001/01 treatment resulted in significant dose-related decreases in IL-6 production for two of the three subjects, Donors A (FIG. 8A) and Donor B (FIG. 8B), equivalent to or greater than the effect of the positive control Birb 796. The sample from Donor C (FIG. 8C) shows reduction of IL-6 by Birb-796 (positive control), but does not show reduction in IL-6 on treatment with SPH-X.

It has been reported that effective treatment of IBD with biologic therapeutic agents is accompanied by reduction in IL-6 levels, and a reduction at week 10 of treatment with a biologic IBD therapeutic (p=0.022) was associated with a sustained clinical response after 12 months of the treatment. Caviglia, et al., J. Clin. Med., vol. 9, 800 (2020). Non-responsive patients did not exhibit lower IL-6 production at the 10-week mark, which was the first post-treatment test reported. Thus, as suggested by this report, not all IBD patients respond to all therapeutics or in the same way. Similarly, Donor C in FIG. 8 may be a subject whose UC that does not respond to CFN001/01, while the subject does appear to respond to Birb 796. CFN001/01 also produced dose-related reductions in the release of the inflammatory cytokines IL-17A and tumor necrosis factor alpha (TNF-α) in some UC patients.

The detailed description set forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method to treat an inflammatory bowel disease in a subject in need of such treatment, which comprises administering to the subject an effective amount of a compound of Formula (I) 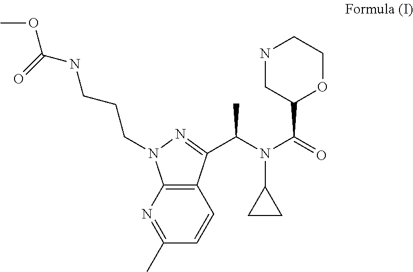
or a pharmaceutically acceptable salt thereof.

2. The method of claim 1, wherein the inflammatory bowel disease is ulcerative colitis.

3. The method of claim 1, wherein the inflammatory bowel disease is Crohn's disease.

4. The method of claim 1, wherein the compound of Formula (I) or a pharmaceutically acceptable salt thereof is administered orally.

5. The method of claim 1, wherein the compound of Formula (I) or a pharmaceutically acceptable salt thereof is administered as a suppository.

6. The method of claim 1, wherein the compound of Formula (I) or a pharmaceutically acceptable salt thereof is administered to the subject at least once per day.

7. The method of claim 6, wherein at least one dose of the compound of Formula (I) or a pharmaceutically acceptable salt thereof is administered to the subject twice daily.

8. The method of claim 1, wherein the dosage of the compound of Formula (I) or a pharmaceutically acceptable salt thereof administered to the subject is between 25 mg and 800 mg.

9. The method of claim 1, wherein the compound of Formula (I) or a pharmaceutically acceptable salt thereof is administered as a delayed release formulation, preferably a formulation that is configured to promote release of the compound of Formula (I) or a pharmaceutically acceptable salt thereof in the lower gastrointestinal tract, or is configured to reduce release of the compound of Formula (I) or a pharmaceutically acceptable salt thereof in the stomach.

10. The method of claim 1, wherein the subject is also treated with at least one additional IBD therapeutic.

11. The method of claim 10, wherein the at least one additional IBD therapeutic is selected from:
a) Anti-TNFα agents;
b) Sphingosine-1-phosphate (S1P)-receptor modulators;
c) Anti-adhesion (anti-integrin) agents;
d) IL-12/IL-23 inhibitors;
e) Transforming growth-factor beta (TGFβinhibitors;
f) Phosphodiesterase 4 (PDE4) inhibitors;
g) Janus kinase (JAK)/signal transducers and activators of transcription (STAT) inhibitors;
h) Stem-cell transplants;
i) Fecal microbiota transplants (FMT);
j) Plasminogen activator inhibitor-1 (PAI-1) inhibitors;
k) Aminosalicylates;
l) Anti-inflammatory corticosteroids; and,
m) Immune pathway inhibitors.

12-30. (canceled)

31. A pharmaceutical composition comprising a compound of Formula (I) 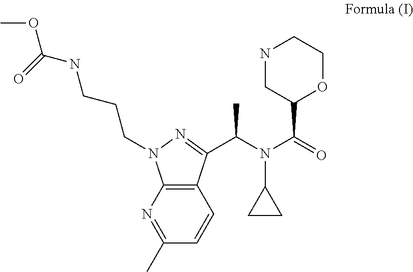
or a pharmaceutically acceptable salt thereof admixed with an additional IBD therapeutic agent.

32. The pharmaceutical composition of claim 31, which is a solid dosage form for oral administration or a suppository.

33. The pharmaceutical composition of claim 31, which comprises between 25 mg and 800 mg of the compound of Formula (I) or a pharmaceutically acceptable salt thereof.

34. The pharmaceutical composition according to claim 31, wherein the compound of Formula (I) or a pharmaceutically acceptable salt thereof is prepared as a delayed release formulation.

35. The pharmaceutical composition according to claim 31, wherein the pharmaceutical composition is configured to promote release of the compound of Formula (I) or a pharmaceutically acceptable salt thereof in the lower gastrointestinal tract, or is configured to reduce release of the compound of Formula (I) or a pharmaceutically acceptable salt thereof in the stomach.

36. The pharmaceutical composition according to claim 31, wherein the at least one additional IBD therapeutic is selected from:
a) Sphingosine-1-phosphate (S1P)-receptor modulators;
b) Transforming growth-factor beta (TGFβ) inhibitors;
c) Phosphodiesterase 4 (PDE4) inhibitors;
d) Janus kinase (JAK)/signal transducers and activators of transcription (STAT) inhibitors;
e) Plasminogen activator inhibitor-1 (PAI-1) inhibitors;
f) Aminosalicylates;
g) Anti-inflammatory corticosteroids; and,
h) Immune pathway inhibitors.

37. The method of claim 10, wherein the at least one additional IBD therapeutic is selected from:
a) an Anti-TNFα agent selected from infliximab, adalimumab, certolizumab, and golimumab;
b) the Sphingosine-1-phosphate (S1P)-receptor modulator ozanimod;
c) an Anti-adhesion (anti-integrin) agent selected from natalizumab, vedolizumab, and ertolizumab;
d) an IL-12/IL-23 inhibitor selected from ustekinumab and risankizumab;
e) a Transforming growth-factor beta (TGFβinhibitor selected from mongersen and pirfenidone;
f) the Phosphodiesterase 4 (PDE4) inhibitor aprimelast;
g) a Janus kinase (JAK)/signal transducer and activators of transcription (STAT) inhibitor selected from tofacitinib and filgotinib;
h) a Stem-cell transplant selected from hematopoietic stem cells and adipose-derived stem cells;
i) Fecal microbiota transplants (FMT);
j) a Plasminogen activator inhibitor-1 (PAI-1) inhibitor selected from MDI-2268 and tiplaxtinin;
k) an Aminosalicylate selected from mesalamine, balsalazide, and olsalazine;
l) Anti-inflammatory corticosteroids; and,
m) An Immune pathway inhibitor selected from azathioprine, mercaptopurine, cyclosporine, and methotrexate.

38. The pharmaceutical composition of claim 36, wherein the at least one additional IBD therapeutic is selected from:
a) ozanimod;
b) mongersen or pirfenidone;
c) aprimelast;
d) tofacitinib or filgotinib;
e) MDI-2268 or tiplaxtinin;
f) mesalamine, balsalazide, or olsalazin;
g) an Anti-inflammatory corticosteroid; and,
h) azathioprine, mercaptopurine, cyclosporine, methotrexate, or a TNF-α inhibitors.
